(19) United States
(12) Patent Application Publication
(10) Pub. No.: US 2022/0369928 A1
(43) Pub. Date: Nov. 24, 2022

(54) CONTACTLESS REAL-TIME STREAMING OF PATIENT VITAL INFORMATION

(71) Applicant: Caremeda LLC, Lakewood Ranch, FL (US)

(72) Inventors: Wilson Paul Ward, Redwing, MN (US); Matthew Hartley, Mount Pleasant, SC (US)

(73) Assignee: Caremeda LLC, Lakewood Ranch, FL (US)

(21) Appl. No.: 17/353,025

(22) Filed: Jun. 21, 2021

Related U.S. Application Data
(60) Provisional application No. 63/191,176, filed on May 20, 2021.

(51) Int. Cl. A61B 5/00; A61B 5/021; A61B 5/0205; A61B 5/024; A61B 5/08; A61B 5/145; G16H 40/67; G16H 80/00; G16H 15/00; G16H 50/20; G16H 30/20; G16H 30/40; G16H 50/30; H04N 7/14; G01N 33/49

(52) U.S. Cl. CPC A61B 5/0022; A61B 5/0077; A61B 5/7264; A61B 5/021; A61B 5/0205; A61B 5/024; A61B 5/0816; A61B 5/14542; G16H 40/67; G16H 80/00; G16H 15/00; G16H 50/20; G16H 30/20; G16H 30/40; G16H 50/30; H04N 7/141; G01N 33/49

(57) ABSTRACT

A method is described which provides patient vital sign measurements to a medical professional during a telemedicine visit. The method includes establishing, by a patient communication device, a video call with a medical professional communication device, processing at least one of a patient video feed from a camera of the patient communication device or an embedded sensor of the patient communication device, determining one or more patient vital sign measurements based on at least one of the processed patient video feed or signal; and transmitting, the one or more patient vital sign measurements and the patient video feed to the medical professional communication device, receipt of the one or more vital sign measurements causing the medical professional communication device to display the one or more vital sign measurements concurrently with the patient video feed.

### CROSS-REFERENCE TO RELATED PATENT APPLICATIONS

This application claims the benefit of priority to provisional U.S. Application No. 63/191,176, filed on May 20, 2021, which is incorporated by reference herein in its entirety.

### BACKGROUND

Telemedicine is the use of electronic communication and information technologies to provide healthcare when distance separates the medical professional from the patient. With the proliferation of video calling via smartphone, tablet, or personal computer, telemedicine has become increasingly popular. Telemedicine can often replace a visit to a medical professional's office, increasing efficiency for the medical professional and providing convenience to the patient. It can also help to isolate the immunocompromised from sick patients who must visit a care facility in person.

However, telemedicine today has certain limitations compared to in-person care. Because the medical professional is not able to physically interact with the patient, it can be difficult to measure vital signs or examine other physical conditions not apparent on a screen. A patient can purchase or be provided with various devices, such as blood pressure cuffs, pulse oximeters, and infrared thermometers, to allow the patient to take his or her own vitals and report them to the medical professional. Many patients may not have access to these devices or may lack the ability to operate the devices on their own. For example, elderly or infirm patients may lack the motor skills or understanding to operate the devices. Additionally, there may be technological challenges in connecting the devices to the telemedicine system to directly report the measurements. For example, the device may not connect properly to the patient's computer or tablet using Bluetooth, a USB port on the patient's computer may be damaged or disabled, or there could be problems installing the software necessary to connect the device to the patient's computer or tablet. It would therefore be advantageous to provide this data to the medical professional during the telemedicine visit without the need for extra hardware or extra effort on the part of the patient.

### SUMMARY OF THE INVENTION

In accordance with some aspects of the disclosure, a method for providing patient vital sign measurements to a medical professional during a telemedicine visit is disclosed. The method may include establishing, by a patient communication device, a video call with a medical professional communication device; processing, by the patient communication device, at least one of a patient video feed from a camera of the patient communication device or a signal from an embedded sensor of the patient communication device; determining, by the patient communication device, one or more patient vital sign measurements based on at least one of the processed patient video feed or the processed signal; and transmitting, by the patient communication device, the one or more patient vital sign measurements and the patient video feed to the medical professional communication device, receipt of the one or more vital sign measurements causing the medical professional communication device to display the one or more vital sign measurements concurrently with the patient video feed.

In accordance with some aspects of the disclosure, a system is disclosed. The system may include one or more hardware processors configured by machine-readable instructions to: establish a video call with a medical professional communication device; process at least one of a patient video feed from a camera of the patient communication device or a signal from an embedded sensor of the patient communication device; determine one or more patient vital sign measurements based on at least one of the processed patient video feed or the processed signal; and transmit the one or more patient vital sign measurements to the medical professional communication device, receipt of the one or more vital sign measurements causing the medical professional communication device to display the one or more vital sign measurements concurrently with the patient video feed.

In accordance with some aspects of the disclosure a non-transitory computer-readable storage medium is disclosed. The non-transitory computer-readable storage medium may have instructions embodied thereon that, upon being executed by one or more processors, cause the one or more processors to perform a method, the method comprising: establishing a video call with a medical professional communication device; processing at least one of a patient video feed from a camera of the patient communication device or a signal from an embedded sensor of the patient communication device; determining one or more patient vital sign measurements based on at least one of the processed patient video feed or signal; and transmitting the one or more patient vital sign measurements to the medical professional communication device, receipt of the one or more vital sign measurements causing the medical professional communication device to display the one or more vital sign measurements concurrently with the patient video feed.

### BRIEF DESCRIPTION OF THE DRAWINGS

FIG. 1A-1C illustrate the limitations of using an auxiliary device to measure patient vital signs.

FIG. 1D illustrates a solution to the limitations of using an auxiliary device to measure patient vital signs, in accordance with one or more implementations.

FIG. 2 illustrates a system configured to transmit contactless patient vital sign measurements during a telemedicine call, in accordance with one or more implementations.

FIG. 3 illustrates a system configured to transmit contactless patient vital sign measurements during a telemedicine call, in accordance with one or more implementations.

FIG. 4 illustrates a process for transmitting contactless patient vital sign measurements during a telemedicine call, in accordance with one or more implementations.

FIG. 5 illustrates a process for transmitting contactless patient vital sign measurements during a telemedicine call, in accordance with one or more implementations.

FIG. 6 illustrates a screen display of a medical professional communication device during a telemedicine call, in accordance with one or more implementations.

FIG. 7 illustrates a screen display of a medical professional communication device during a telemedicine call, in accordance with one or more implementations.

FIG. 8 illustrates a process for transmitting contactless patient vital sign measurements during a telemedicine call, in accordance with one or more implementations.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

In a telemedicine visit, a medical professional and patient may communicate via video calling. As used herein, the term “medical professional” refers to physicians, nurses, medical technicians, psychiatrists, psychologists, or other personnel trained to work in medical environments. The medical professional's communication device may communicate with the patient's communication device over a network. The communication devices may be smartphones, tablets, personal computers, or other devices containing a camera, speaker, a microphone, and a display screen and capable of transmitting and receiving audiovisual data over a computer network. The camera of the communication device generally faces the direction of the screen, such that the user's face may be captured by the camera while the user looks at the screen.

The screen of the medical professional's communication device may display a real-time video feed from the patient's camera, and the speakers of the medical professional's communication device may project a real-time audio feed from the patient's microphone. Similarly, the screen of the patient's communication device may display a real-time video feed from the medical professional's camera, and the speakers of the patient's communication device may project a real-time audio feed from the medical professional's microphone. As used herein, the term “real-time” may mean instantaneously or with a brief delay due to processing and transmission processes. The medical professional's communication device may be configured to only receive live patient vital sign measurements during a telemedicine call, such that, for example, the live vital sign measurements may be automatically matched to the correct patient video feed. Though it is preferable for each device used in a video call to have a camera, some of the embodiments disclosed herein do not necessarily require the medical professional's communication device to have a camera.

Because the medical professional is not able to physically interact with the patient during a telemedicine visit, the patient must collect his or her own vital sign measurements if the medical professional requires them. As used herein, the term “vital signs” or “vitals” refer to any measurable indicia of patient health, including, but not limited to heart rate, respiratory rate, blood pressure, and blood oxygen level. Various devices may be used to measure vital signs, including blood pressure cuffs, heart rate monitors, and pulse oximeters. However, a patient may not have access to or the ability to use such devices. In the embodiments disclosed herein, systems and methods for contactless measurement, transmission and display of vital sign data are provided in which the patient may not need to take any action beyond facing the camera of his or her communication device.

Currently, telemedicine patients have two general methods of transmitting their vital signs to their doctors. First, the patient can measure his or her own vital signs using a variety of techniques or devices and can manually report the results to the doctor, for example, via email or verbally during a telemedicine visit. The first method requires patients, who may be elderly or unwell, to use devices or methods that they may not have the knowledge, dexterity, or training to use. This carries a high likelihood of faulty measurements that could affect the doctor's judgement in evaluating the patient. Further there could be errors in the reporting of the measurements to the doctor. The patient may misread the device or his own handwriting, the patient could misspeak, or the doctor could mishear the patient if the results are reported verbally. If the doctor is required to observe the patient taking the measurements during the telemedicine call, measuring vitals this way could cause unnecessary delay and require the doctor to rush through the rest of the call.

A second current method of transmitting vital signs to a doctor requires a patient to use an auxiliary device connected to their computer, tablet, or smartphone to measure the vitals and send the results to their doctor using software associated with the device. This solves some of the potential communication problems of the first method, as the measurements may be automatically reported as they are taken, but this method still has drawbacks. First, patients may not own or have access to these devices. Many patients are using telemedicine for the first time or use it so infrequently that they would not consider purchasing one of these devices. Next, while these devices have been designed to be more consumer-friendly than professional medical devices, some patients may still struggle to use them properly, resulting in faulty measurements. For example, a patient may not place the device on his or her body in the correct position or orientation or for long enough to get an accurate reading. Furthermore, these devices must have a data connection to the patient's computer, tablet, or smartphone, such as a Bluetooth, Wi-Fi, or wired connection. Less tech-savvy patients may have trouble setting up these connections, or there may be hardware or software issues that prevent the connection entirely. For example, while it may be useful for a patient to transmit live vital measurements during a telemedicine call while the doctor observes. The connection between the auxiliary device and the patient's computer, tablet, or smartphone may cause latency between the patient's video feed and the vital measurements. This latency may cause a significant lag between the vital data that is shown on the user interface at the professional medical and the video feed.

The methods of the present disclosure may provide immediate vital sign measurements without the need for confusing auxiliary devices or additional technological knowhow on the part of the patient. Patient vitals can be measured without any action on the part of the patient beyond joining and participating in the telemedicine call. Contactless vital measuring technology can be built into the patient's telemedicine software. Using, for example, transdermal optical imaging, the video feed captured by the camera of the patient's computer, smartphone, or tablet can be processed by the software to determine the patient's vital sign measurements. The can patient simply join the telemedicine call, and his vital signs are measured while he speaks naturally to the doctor. Since no auxiliary devices are needed, this method is available to every patient that is able to join a video call, and there is no need to struggle with setting up the data connection between the device and the patient's computer, tablet, or smartphone. The risk of faulty measurements due to an error by the patient in operating an unfamiliar device is also eliminated. Further, because the patient's computer, tablet, or smartphone is taking the measurements, there may be no latency caused by the connection of an auxiliary device. Patient vital signs can be sent in real-time along with the video feed, allowing the doctor to observe any changes as they happen.

Another advantage of the methods of the present disclosure is that several vital measurements can be taken simultaneously and seamlessly. Auxiliary devices may require each measurement to be taken sequentially. For example, the patient may have to take a blood pressure reading, then a heart rate reading, then a respiration rate reading. The patient's discussion with the doctor may be interrupted during the time the patient spends taking the measurements. With the contactless technology disclosed herein, the patient can have a natural conversation with the doctor without stopping to take measurements. The patient may participate in the call as if he did not even know measurements were being taken.

The patient and medical professional may interact within a patient application (“app”) and a medical professional app, respectively, on their respective devices. The patient and medical professional may log in to their respective apps to facilitate the telemedicine call, access their respective schedules, send messages to each other, and view the patient's medical information. Additionally, the medical professional may send the patient surveys asking the patient to self-report their subjective wellbeing or to record vital sign measurements outside of a telemedicine visit. For example, the medical professional may ask the patient to record his or her blood pressure each day for the week leading up to a telemedicine visit, which the patient can measure using the contactless technology. The surveys may also ask the patient to self-report data not captured by the contactless technology, such as the number of daily walking steps taken or hours of nightly sleep. These may be entered manually or by syncing an external device with the patient app. The patient may also log health data without being prompted by a survey sent by the medical professional for his or her own records. The apps may display multiple measurements on a chart to show how a certain vital sign changes over time. The medical professional may configure the medical professional app to send alerts to the medical professional and/or the patient when a measurement submitted by the patient falls outside of a certain range. For example, the medical professional may receive an alert when a patient's blood pressure falls outside of the healthy range for a patient's age. Both apps may be configured to allow the user to add the patient's family members to the patient's profile. Family members may be allowed to join in on telemedicine calls with the patient and the medical professional and may have limited or unlimited access to the patient's medical records if the patient permits.

A “back office” app can also be used by, for example, medical administrative staff, to add patient, family member, and medical professional profiles to the system. In the “back office” app medical professionals can be assigned to patients and care facilities. Telemedicine appointments can be scheduled for the medical professional. The administrative staff can also add family members to the patient's profile and set up health alerts as described above.

All electronic communications sent and received within the disclosed systems and methods can be encrypted to comply with the requirements of the Health Insurance Portability and Accountability Act (HIPAA) in order to protect confidential patient information from interception by or dissemination to third parties. This may include the video and audio communications as well as vital sign measurements and any textual communication sent within the apps.

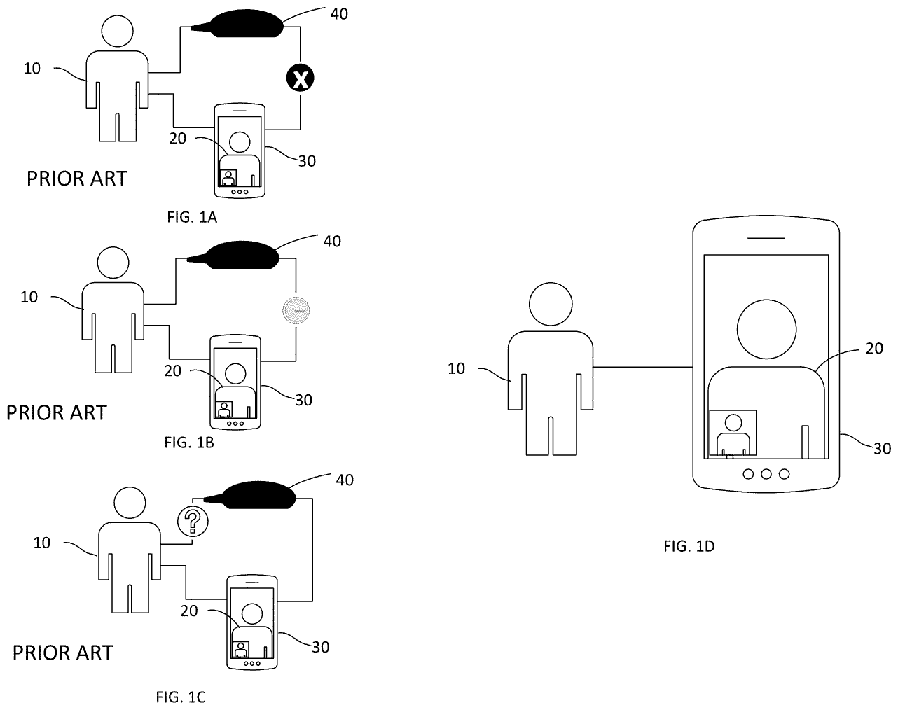

Referring to FIGS. 1A-1C, several drawbacks of using an auxiliary device to measure vitals during a telemedicine call are illustrated. FIG. 1A shows a patient 10 during a telemedicine call with a medical professional 20 on the patient communication device 30. The patient communication device 30 may be, for example, a phone, tablet, or personal computer. The medical professional 20 has instructed the patient 10, to measure his own vitals using the auxiliary device 40. The auxiliary device is 40 is meant to have a data connection with the patient communication device 30, for example, a Bluetooth, Wi-Fi, or wired connection. However, the auxiliary device 40 is unable to connect to the patient communication device 30, either due to hardware or software problems, or because the patient 10 was not able to set up the auxiliary device connection properly. In this circumstance, the telemedicine visit would have to continue without the medical professional 20 receiving the patient's vitals, making it more difficult, if not impossible, for the medical professional to provide complete and accurate medical advice.

FIG. 1B shows another potential problem resulting from the use of the auxiliary device 40 during a telemedicine call. Here, there is latency in the connection between the auxiliary device 40 and the patient communication device 40. This latency could be caused by interference with a Bluetooth connection or simply slow upload and download speeds from the auxiliary device 40 and the patient communication device 30, respectively. Thus, the medical professional 20 may receive the measurements several seconds behind the video feed of the patient 10. While, in most cases, a delay in the delivery of vital sign measurements to the medical professional 20 may be harmless, there may be circumstances in which a medical professional 20 wishes to observe changes in the patient's vitals in real-time while they watch the patient's video feed. Added latency from the auxiliary device could prevent this type of observation.

FIG. 1C shows a third potential problem with using an auxiliary device 40 to measure vital signs during a telemedicine call. The patient may not understand how to use the auxiliary device 40 properly. This can cause a number of problems that could potentially have harmful effects on the patient's health. For example, neither the patient 10 nor the medical professional 20 may realize that the patient 20 is not using the auxiliary device 40 properly. If the auxiliary device 40 reports incorrect measurements to the medical professional 20 due to an error by the patient 10, the medical professional 20 may suggest a course of treatment based on erroneous data that could harm the patient. For example, if the auxiliary device 40 reports that the patient 10 has high blood pressure due to an error in using the auxiliary device 40 by the patient 10, the medical professional 20 may prescribe an ACE inhibitor. If the patient 10 in fact has normal or low blood pressure, taking an ACE inhibitor could cause dizziness or fainting. This is just one example of one of the many risks of inaccurate readings that could result from the patient 10 performing his or her own vital sign measurements with an auxiliary device 40.

If the patient 10 or the medical professional 20 realizes that the patient 10 does not understand how to use the auxiliary device 20, the medical professional 20 may have to take time during the telemedicine call to instruct the patient 10. This could cause delays in the medical professional's schedule or a reduction in the amount of time the patient 10 has to discuss his health with the medical professional 20. If the patient 10 is unable to understand how to use the auxiliary device 40 even after being instructed, the medical professional 20 may not be able to get vital measurements at all. In this case, the medical professional 20 will need to evaluate the patient 10 with incomplete information, or the patient 10 will need to visit the medical professional 20 in person, losing the benefits of using telemedicine.

FIG. 1D is an illustration of a patient 10 using the patient communication device 30 to measure his vital signs using transdermal optical imaging or other technology not requiring a separate device, including but not limited to imaging technologies, during a telemedicine call with a medical professional 20, according to an embodiment of this disclosure. Since no auxiliary device is needed to measure the vital signs, the problems with connecting such a device to the patient communication device 20 and any delay caused by the connection are eliminated. Furthermore, there is no risk that the patient 10 will not understand how to take the measurements. Vital measurements can be initiated by the patient 10, the medical professional 20, or automatically as a function of the telemedicine software. The patient 10 may only need to be able to join and participate in the telemedicine video call, and his or her vitals may be measured without any additional technological understanding or auxiliary devices.

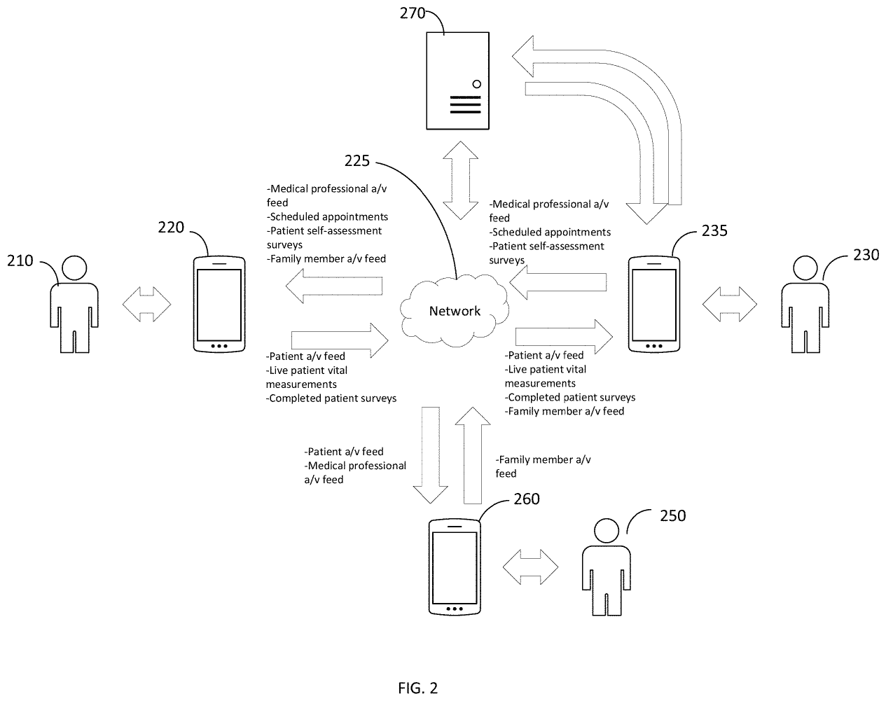

Referring to FIG. 2, a system is shown illustrating the capturing and reporting real-time vitals during a telemedicine call according to an embodiment of this disclosure. A patient 210 can use a patient communication device 220 to connect to a video call with a medical professional 230 via a network 225 (e.g., via a HIPAA-compliant encrypted network data connection). A medical professional 230 can use a medical professional communication device 235 to connect to the telemedicine call with the patient 210 via the network 225. The network 225 can be configured to allow audio and video feeds and other information from the patient communication device 220 to be transmitted to the medical professional communication device 235 and vice versa. The communication devices 220, 235 may be in communication with an electronic medical records (EMR) system 270 via the network 225. Alternately or additionally, the medical professional communication device 235 may directly communicate with EMR system 270 via a separate network connection, such as a local area network. The network may allow both video call data and vital sign measurements to be transmitted, or the network 225 may include more than one network connections.

The system may optionally include one or more family members 250 using one or more family communication devices 260 to connect to the telemedicine call with the patient 210 and the medical provider 230 via the network 225. Patient family members 250 may log into a family member app on family member communication devices 260. The patient 210 may grant permission to family members 250 to join the patient 210 on telemedicine calls with the medical professional 230. The family communication devices 260 may transmit and receive video and data streams to and from the patient communication device 220 and the medical professional communication device 235. The patient 210 may also grant permission to family members 250 to access all or a portion of the patient's medical records, and may grant permission to have patient vitals transmitted to the family member communication devices at the same time they are transmitted to the medical professional device 235 during a telemedicine call. The system may optionally measure family member vital signs if the family member 250 gives consent to have his or her vitals measured. For example, the family member app may function similarly to the patient app, enabling the family member to measure and send his or her vitals by making a selection within the app. The app may process the video feed from the family member communication device 260 and/or signals from one or more embedded sensors of the family member communication device 260 to determine the family member vital sign measurements and transmit the measurements to the medical professional communication device 235 concurrently or separately from the vital measurements sent by the patient communication device 220. Family member vital sign measurements may optionally be transmitted to the patient communication device 220 as well. Family member vital signs may be overlaid on the video feed from the family member communication device 260 and displayed on the medical professional communication device 235 and/or the patient communication device 220. The system may optionally include additional medical professionals joining the call with the medical professional 230 in person or via additional medical professional communication devices.

The patient communication device 220 may be configured to process the patient's video feed using transdermal optical imaging and/or to use other contactless measuring methods to measure the patient's vital signs. Once the vital sign measurements are determined, the patient communication device 220 can transmit the data to the telemedicine system 225 via a network. The telemedicine system 225 then can transmit the data to the medical professional communication device 235 via a network, where it can be displayed on the medical professional communication device's screen. The data may be overlaid as text on top of the video feed in a “heads-up display” fashion.

Transdermal optical imaging is a method of isolating hemoglobin concentration from raw images taken from a digital camera. Light travels beneath the patient's skin and re-emits after travelling through different skin tissues. The re-emitted light may then be captured by the camera of the patient communication device 210. The dominant chromophores affecting the re-emitted light are melanin and hemoglobin. Since melanin and hemoglobin have different color signatures, it has been found that it is possible to obtain images mainly reflecting hemoglobin concentration under the epidermis. The raw images are processed by an image processing module within the patient app of the patient communication device 220.

Using transdermal optical imaging, the image processing module within the patient app may obtain each captured image in a video stream and perform operations upon the images to generate a corresponding optimized hemoglobin concentration image of the patient. From the hemoglobin concentration data, the facial blood flow localized volume concentrations can be determined. Machine learning training datasets can be generated by comparing processed transdermal optical images to known vital measurements taken by traditional methods, such as a blood pressure cuff or an electrocardiogram and labeling the images with the correct measurements. Once the datasets are generated, they can be used to train a machine learning model to be capable of producing reliable measurements of vital signs based on a processed video feed. Possible vital sign measurements that may be produced include, but are not limited to, blood pressure, heart rate, and blood oxygen saturation. The model may then be loaded into the patient app to allow for vital sign measurement using the video feed from the camera of the patient communication device 220 as the camera captures video and the patient communication device 220 transmits the video to the medical profession device 235. A full disclosure of the transdermal optical imaging process can be found, for example, in U.S. Pat. No. 10,888,256, the entire disclosure of which is incorporated by reference herein.

In some embodiments, the patient app may measure the patient's vital signs during the call only when the patient remains within a predetermined portion of the screen on the patient communication device 220 and/or predetermined view of the camera of the patient communication device 220. Such may be the case when the machine learning model is trained to determine vitals of patients within a certain area of an image and may enable the machine learning model to be more accurate with its predictions. For example, over the course of a telemedicine call, the patient may move around relative to the direction the camera on the patient communication device 220 is pointing. There may be certain locations where the patient may be depicted on images where the machine learning model may not be able to accurately predict vital sign measurements of the patient. When a patient is captured in such locations, which the patient communication device 220 and/or the app may determine using object character recognition techniques, the patient app may generate and/or present an alert at the patient communication device 220 indicating for the patient to appear back within view of the camera. Thus, during the call, the app on the patient communication device 220 may continue to capture accurate vital sign data of the patient.

It should be understood that the systems and methods claimed in this application are not limited to the use of transdermal optical imaging. Any technology that can be integrated and/or embedded into the patient communication device 220 and can be operated without requiring the patient 210 to wear, connect, or apply any external devices is contemplated by this disclosure. For example, the patient communication device 220 may include one or more embedded sensors, such as a pressure sensor, an infrared camera, or a radar sensor or may use traditional image processing techniques to measure patient vitals. The patient communication device 220 and/or patient app may be configured to process a signal from an embedded sensor of the patient communication device 220 in order to determine patient vital sign measurements. An important advantage of the present disclosure over previous systems is that the patient can operate the patient communication device 210 as he or she would for any video call. The patient vital signs may be measured and reported to the medical professional 230 without any additional action by the patient 210.

In some implementations, for example, the patient communication device 220 may include an infrared camera (e.g., an embedded infrared camera). The infrared camera may be embedded next to the optical camera of the patient communication device facing the same direction of the screen. This may allow the infrared camera to capture an image or signal of the infrared energy emitted by the patient, as well as anything within the frame of the infrared camera, while the patient looks at the screen. The patient communication device 220 and/or the patient app may be configured to process this image or signal from the infrared camera to identify the heat signature of the patient and isolate it from the background. After identifying the patient in the infrared image, the patient communication device and/or the app may determine, for example, the patient's body temperature or other vital signs (e.g., the patient communication device and/or the patient app may determine the patient's temperature based on the amount of infrared energy that is focused around individual pixels of the infrared sensors such that higher amounts of energy correspond to higher temperatures). This can be advantageous as it can allow the patient's body temperature to be reported to the medical professional during a telemedicine call without the patient having to interrupt the call to find an oral thermometer or to stop talking while using an oral thermometer.

The patient communication device 220 may alternatively or additionally include one or more radar sensors (e.g., embedded radar sensors). A radar sensor may be embedded into the patient communication device 220 such that it broadcasts a radio signal towards the patient during a telemedicine call and detects the modulation of the signal caused by the patient. The modulation may vary based on the patient's breathing and heartbeat. The patient communication device 220 and/or the patient app may process this signal in order to determine, for example, the patient's respiratory rate, heart rate, or other vital sign measurements. Measuring vitals from a radar sensor provides many of the same advantages as processing a video feed from a camera using transdermal optical imaging. The patient's discussion with the doctor may be uninterrupted, the measurements may be accurately reported, and no auxiliary devices may be needed. Radar sensors may also be advantageous if the lighting conditions prevent the patient communication device from receiving a clear enough image to perform the transdermal optical imaging process.

The processing of the video feed from the camera and/or the signals from the embedded sensors may take place by the patient app on the patient communication device 220. Because the video call and the processing of the video feed and signals may be performed within a single app, latency between the video feed and the vitals measurements arriving at the medical professional communication device can be minimized (e.g., any latency caused by the transfer of data between applications or devices may be minimized).

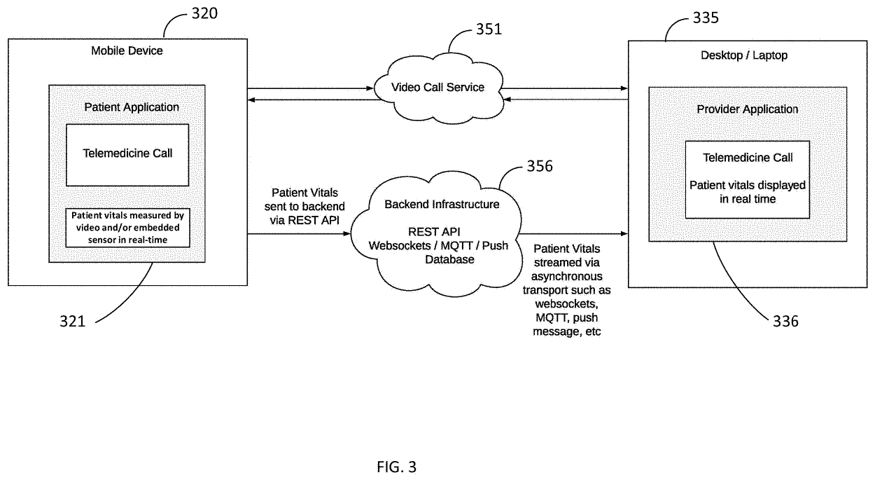

FIG. 3 illustrates the communication between the patient communication device 320 and the medical professional communication device 335 during a telemedicine call according to an embodiment of this disclosure. The patient device 320 is shown as a “mobile device” and medical professional device is shown as a “desktop/laptop.” However, either of the devices may be a mobile device, such as a smartphone or tablet, a personal computer, such as a desktop or laptop, or any other device. The devices 320, 335 may connect to the video call service 351 which facilitates the telemedicine call by transmitting the audiovisual feed from the patient communication device 320 to the medical professional device 335 and vice versa. The video call service 351 may be a service that operates on the devices 320 and/or 335 that enables the devices 320 and 335 to communicate with each over a network (e.g., a telephony network, a cellular network, or the Internet).

The patient can access the telemedicine call via the patient app 321, and the medical professional can access the telemedicine call via the provider app 336. During the telemedicine call, the patient app 321 can measure patient vitals by processing the video feed from the patient communication device 320 and/or by processing signals from one or more embedded sensors of the patient communication device 320. For example, the patient application 321 may process the video feed using transdermal optical imaging to determine patient vital sign measurements. Alternatively or additionally, the patient application 321 may process a signal from an embedded sensor such as an infrared camera or a radar sensor to determine patient vital sign measurements. The patient communication device 320 can transmit the vital measurements to the backend infrastructure 356 via an API (e.g., a REST API). The video call service 351 and the backend infrastructure 356 may be connections over separate networks or may be separate connections on a single network. The backend infrastructure 356 can stream the patient vital measurements to the medical professional communication device 335 via asynchronous transport such as Web Sockets, MQTT, or push message separately and/or concurrently with the audiovisual feed being streamed via the video call service 351. Upon receipt of live vital sign data from a patient communication device 320, the provider application 336 may determine whether the provider application 336 is also receiving and displaying a video feed from the patient communication device 320, indicating that a video call is in progress. For example, the provider application 336 may determine the provider application 336 is exchanging a video feed with the patient communication device 320 responsive to identifying the port of the provider communication device 335 that is connected to the patient application 321. If the provider application 336 cannot identify a port that is connected to a patient communication device or application, the provider application 336 may discard the vital sign data. If the provider application 326 determines that it is receiving and displaying a video feed from the patient communication device, indicating that a video call is in progress, the provider application 336 can combine the video feed and the patient vital measurement into a single display feed, allowing the medical professional to observe the patient and the vital sign measurements simultaneously. In some embodiments, the vital sign measurements may be transmitted as raw numerical values by the patient communication device 320. In this situation, the medical professional communication device 335 may create a graphical representation of the values (e.g., as text or a graph) and overlay it onto the patient video feed. In other embodiments, the patient communication device 320 may create and transmit a graphical representation of the raw numerical values. In this situation, the medical professional communication device 335 may simply overlay the graphical representation on the patient video feed.

For example, a patient may have a telemedicine appointment with a medical professional. The patient may initiate the telemedicine appointment through the patient app 321 by initiating a video call with the medical professional's provider app 336. The patient app 321 and the provider app 336 may communicate with each other via the video call service 351. While initiating the telemedicine appointment, the patient app 321 may establish a second connection (e.g., an API connection with the provider app 336) by sending a connection request to the provider app 336. During the video call, the patient app 321 may receive a video feed captured by a camera of the patient's device 320 and/or a signal from one or more embedded sensors of the patient communication device 320. The patient app 321 may process the video feed and/or the signals received from the one or more embedded sensors as described herein to determine the patient's vital signs during the call. After processing the video feed and/or the signals, the patient app 321 may forward the video feed to the provider app 336 over the video call service 351. Separately, the patient app 321 may transmit the vital sign data that the patient app 336 determined from the video feed and/or the signals to the provider app 336 via the second connection. The provider app 336 may receive the video feed and the vital signs and combine the two sets of data as described herein. The provider app 336 may then present the combined data to the medical professional at a display on the medical professional's device 335, thus providing the medical professional with a real-time view of a patient's vital signs during a telemedicine call.

In alternative embodiments, the patient vitals may be combined with the video feed from the patient communication device 320 by the patient application 321 prior to being sent to the medical professional device 335. The patient device 320 may create a graphical representation of the raw numerical values and overlay the graphical representation of the raw measurement values on the patient video feed. The combined feed may then be streamed to the medical professional device 335 with the vital measurements overlaid on the video feed (e.g., as text or as a graph). In this situation, the medical professional communication device 335 may receive only a combined video feed containing the patient video feed with the overlaid graphical representation of the measurement values, such that the medical professional communication device 335 may not need to combine multiple data streams.

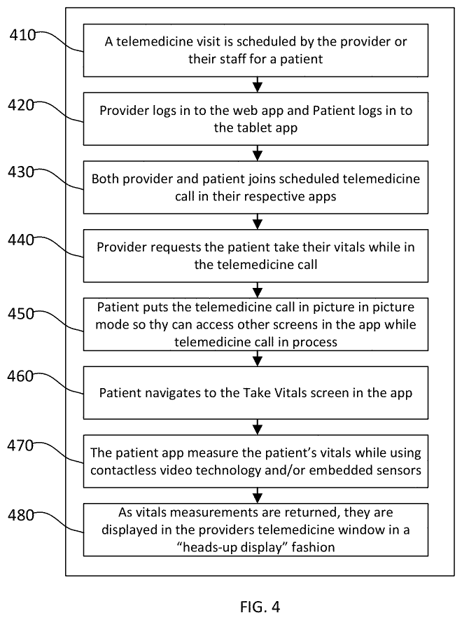

Referring to FIG. 4, an example embodiment of the disclosure is provided which illustrates the operations that may be performed to take, transmit, and display vital sign measurements without the use of additional equipment beyond the communication devices required for a telemedicine visit. At operation 410, a telemedicine visit may be scheduled by the medical professional or the medical professional's staff. At operation 420, the medical professional and the patient may log in to their respective applications (apps) on their communication devices in order to access the telemedicine software. The medical professional's app may contain the medical professional's telemedicine call schedule and patient medical records. At operation 430, the medical professional and the patient may join the scheduled telemedicine call within their respective apps. The medical professional's communication device may receive an audiovisual feed from patient's communication device and the patient's communication device may receive an audiovisual feed from the medical professional's communication device. At operation 440, the medical professional may request that the patient take their vital sign measurements. At operation 450, the patient may put the telemedicine call in picture in picture mode to access other portions of the app during the call. At operation 460, the patient may navigate to the “Take Vitals” screen within the app activates the vital sign measurement system. At operation 470, using contactless video technology such as transdermal optical imaging, the patient's app can measure the patient's vital signs by processing the video feed from patient's communication device. Alternatively or additionally, the patient app can measure the patient's vital signs by processing signals from one or more embedded sensors. At operation 480, the vital sign measurements may be transmitted to the medical professional's communication device and displayed on the screen. The measurements may be displayed in a “heads-up display” fashion, meaning that the measurements are overlaid as text on the video feed of the patient.

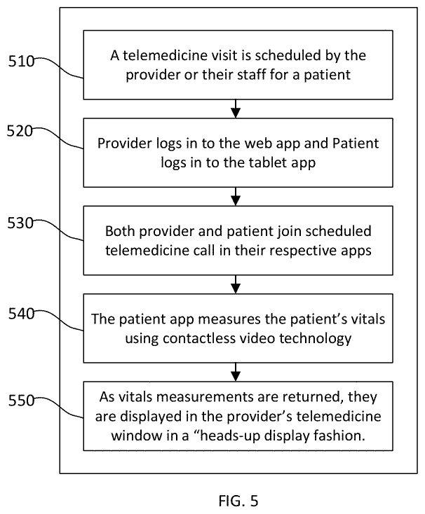

Referring to FIG. 5, a second example embodiment of the disclosure is provided, showing a method in which the patient is not required to take any actions in order to record and transmit vitals to medical professional. At operation 510, a telemedicine visit may be scheduled by the medical professional or the medical professional's staff. At operation 520, the medical professional and the patient may log in to the respective apps on their communication devices in order to access the telemedicine software. At operation 530, the medical professional and the patient may join the scheduled telemedicine call within their respective apps. At operation 540, the patient app can measure the patient's vital signs by processing the video feed from the patient's communication device using contactless video technology such as transdermal optical imaging and/or by processing signals from one or more embedded sensors of the patient's communication device. The measurement may begin with or without any actions being taken by the patient or the medical professional (e.g., the measurements may begin without the patient or the medical professional selecting a button on the screen to cause for the measurements to be taken or by using an auxiliary device to take the measurements). At operation 550, the vital sign measurements may be transmitted to the medical professional's communication device and displayed on the screen. Again, the measurements may be shown on the medical professional's screen as a “heads up display,” overlaid as text on the video feed of the patient.

Any of the above methods may optionally include one or more approved family members joining the telemedicine call with the patient and medical professional. As used herein, “family member” refers to any person approved by the patient to attend the telemedicine call and is not limited to biological or legal members of the patient's family. Additional medical professionals may also join the telemedicine call, either in person with the first medical professional or using additional medical professional communication devices.

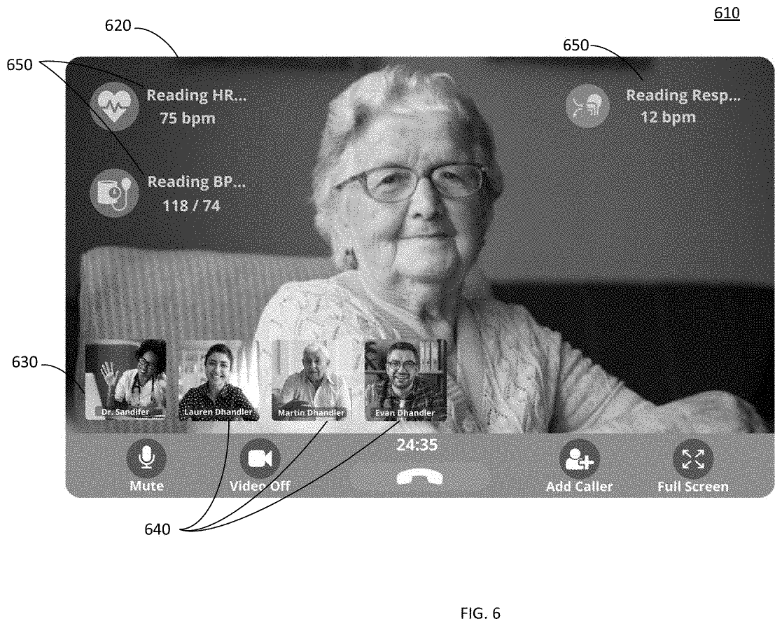

FIG. 6 is an illustration of an example screen display 610 of the medical professional communication device 240 during a telemedicine call with a patient. The video feed 620 from the patient communication device 220 may occupy all or a portion of the screen display 610. The video feed(s) 630 from the medical professional communication device(s) and/or the video feed(s) 640 from the family communication device(s) may be displayed in a separate portion of the screen or as a picture-in-picture on top of the patient video feed 620. The patient vital signs 650 may be displayed in a “heads-up display” fashion over the sides and/or corners of the patient video feed 620. Vitals may be measured, transmitted, and displayed on the screen in real-time without pausing or interrupting the telemedicine call, and without requiring any additional devices or action by the patient. The camera of the patient communication device may be in close proximity to and face the same direction as the screen. As the patient looks at the screen of the patient communication device, the camera may capture images of the patient's face, process the images using transdermal optical imaging, and determine the vital sign measurements. Alternatively or additionally, patient communication device 220 may receive signals from one or more embedded sensors pointed towards the patient as the patient looks at the screen and process the signals to determine the patient vital sign measurements. This can allow the patient to participate in the telemedicine call as he or she would any other video call in which vital signs are not being measured. The measurements may take place entirely in the background while the patient communicates with the medical professional.

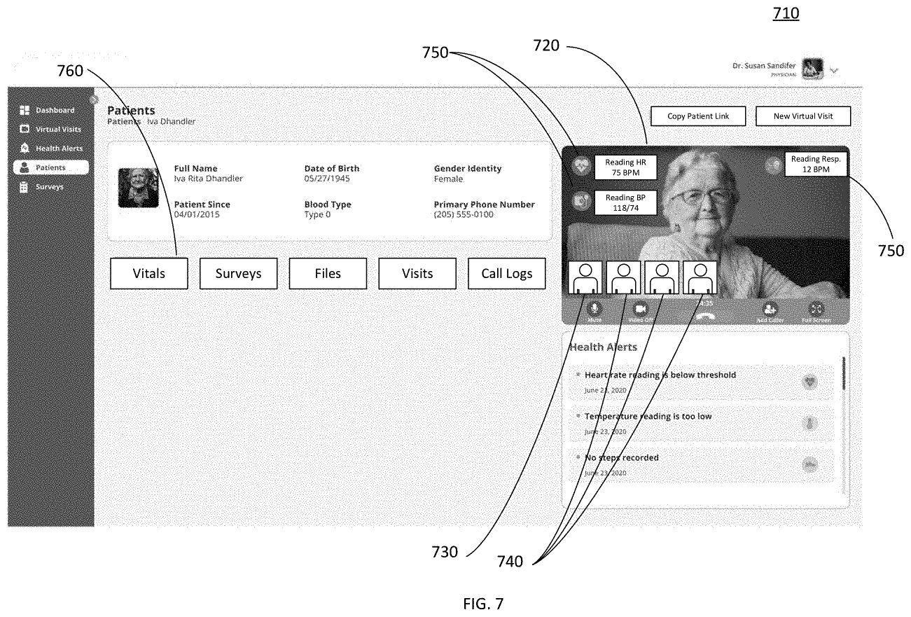

FIG. 7 is an illustration of an alternate example screen display 710 of a medical professional communication device during a telemedicine call with a patient. The patient video feed 720 may occupy a smaller portion of the screen display 710 than as shown in FIG. 5. The video feed(s) 730 from the medical professional communication device(s) and/or the video feed(s) 740 from the family communication device(s) may be displayed in a separate portion of the screen or as a picture-in-picture on top of the patient video feed 720. The patient vital signs 750 may be displayed in a “heads-up display” fashion over the sides and/or corners of the patient video feed 720. Additionally or alternatively, the patient vital signs may displayed on a different portion of the screen, such as below the “Vitals” tab 760. The screen display 710 also may also display various menu options, patient data, patient health alerts, the medical professional's telemedicine calendar, or other data. The screen display may also include text entry fields for the medical professional to take notes during the telemedicine call.

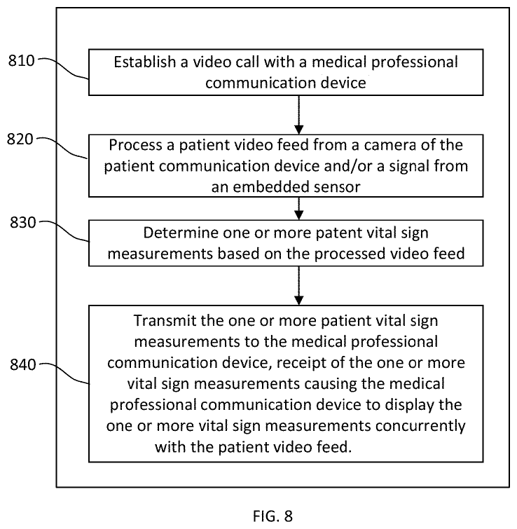

FIG. 8 illustrates an example embodiment of a method of the present disclosure. The listed operations may be performed by a patient communication device. At operation 810, the patient communication device may establish a video call with a medical professional communication device. At operation 820, the patient communication device may process a video feed from a camera of the patient communication device or a signal from a sensor of the patient communication device. Processing the video feed may include evaluating the feed using transdermal optical imaging. The embedded sensor may be, for example, an infrared camera or a radar sensor. At operation 830, the patient communication device may determine one or more vital sign measurements based on the processed video feed. The one or more patient vital signs may include patient blood pressure, patient heart rate, patient respiratory rate, and patient blood oxygen level.

At operation 840, the patient communication device may transmit the one or more vital sign measurements and the patient video feed to the medical professional communication device, causing the medical professional communication device to display the one or more vital sign measurements concurrently with the patient video feed. The patient communication device may also transmit the video feed to the medical professional communication device. The one or more patient vital sign measurements may be overlaid on the patient video feed and displayed on a screen of the medical professional communication device. The one or more patient vital sign measurements may be displayed on the medical professional communication device in real-time along with the video feed, for example, as text or a graph. The patient communication device may combine the patient video feed with the one or more vital sign measurements to create a combined video feed prior to transmitting. Alternatively, the one or more patient vital sign measurements may be transmitted separately from the patient video feed and combined and displayed by the medical professional communication device. The patient video feed may be displayed on a separate portion of the screen of the medical professional device than the one or more patient vital sign measurements.

The processing of the video feed or the signal to determine vitals may be initiated by the patient or the medical professional, e.g., by selecting a menu option within their respective apps on their respective communication devices. The processing may alternatively be initiated automatically by the app at a predetermined time during the video call or continuously for the duration of the video call. In order to process the video feed to measure the patient vital signs, he patient app software on the patient communication device may first have to identify the patient's face in the patient video feed. The app may request that the patient center the camera on the patient's face to begin processing the video feed to determine the vital sign measurements. Alternatively, the app may be able to identify the patient's face in any location within the video feed.

The various illustrative logical blocks, modules, circuits, and algorithm operations described in connection with the examples disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The hardware used to implement the various illustrative logics, logical blocks, modules, and circuits described in connection with the examples disclosed herein may be implemented or performed with a general purpose processor, a DSP, an ASIC, an FPGA or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In some examples, the functions described may be implemented in hardware, software, firmware, or any combination thereof. If implemented in software, the functions may be stored as one or more instructions or code on a non-transitory computer-readable storage medium or non-transitory processor-readable storage medium. The operations of a method or algorithm disclosed herein may be embodied in a processor-executable software module which may reside on a non-transitory computer-readable or processor-readable storage medium. Non-transitory computer-readable or processor-readable storage media may be any storage media that may be accessed by a computer or a processor. For example but not limitation, such non-transitory computer-readable or processor-readable storage media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable storage medium and/or computer-readable storage medium, which may be incorporated into a computer program product.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances, where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Further, unless otherwise noted, the use of the words “approximate,” “about,” “around,” “substantially,” etc., mean plus or minus ten percent.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

1. A method for providing patient vital sign measurements to a medical professional during a telemedicine visit, the method comprising:
establishing, by a patient application on a patient communication device, a video call with a medical professional communication device;
receiving, by the patient application, a first portion of a video feed of a patient's face from a camera of the patient communication device;
subsequent to receiving the first portion, receiving a second portion of the video feed of the patient's face from the camera of the patient communication device;
processing, by the patient application, the first portion of the video feed from the camera of the patient communication device to determine one or more first patient vital sign measurements using one or more image processing techniques as the patient application transmits the first portion of the video feed to the medical professional communication device; and
transmitting, by the patient application, the one or more first patient vital sign measurements and the second portion of the video feed to the medical professional communication device, receipt of the one or more first patient vital sign measurements causing the medical professional communication device to display the one or more first patient vital sign measurements concurrently with the second portion of the video feed,
wherein transmitting the one or more first patient vital sign measurements to the medical professional communication device comprises causing the medical professional communication device to display the one or more first patient vital sign measurements overlaid on the second portion of the video feed,
wherein the one or more first patient vital sign measurements are transmitted as raw numerical values or as a graphical representation.

2. (canceled)

3. The method of claim 1, wherein transmitting the one or more first patient vital sign measurements comprises causing the medical professional communication device to display the one or more first patient vital sign measurements in real-time.

4. The method of claim 1, further comprising combining, by the patient communication device, the one or more first patient vital sign measurements with the second portion of the video feed, such that the one or more first patient vital sign measurements are overlaid on the second portion of the video feed, prior to transmitting the one or more first patient vital sign measurements.

5. The method of claim 1, wherein transmitting the one or more first patient vital sign measurements to the medical professional communication device comprises causing the medical professional communication device to combine the one or more first patient vital sign measurements with the second portion of the video feed such that the one or more first patient vital sign measurements are overlaid second portion of the on the video feed before being displayed on the medical professional communication device.

6. The method of claim 5, wherein transmitting the second portion of the video feed comprises transmitting second portion of the video feed via a first connection, and wherein transmitting the one or more first patient vital sign measurements to the medical professional communication device further comprises establishing a second connection with the medical professional communication device and transmitting the one or more first patient vital sign measurements over the second connection.

7. (canceled)

8. The method of claim 1, wherein the graphical representation comprises one or more of text or a graph.

9. The method of claim 1, wherein the patient communication device further comprises an infrared camera or a radar sensor configured to determine additional patient vital sign measurements.

10. The method of claim 1, wherein processing the first portion of the video feed comprises analyzing the first portion of the video feed using transdermal optical imaging.

11. The method of claim 10, wherein using transdermal optical imaging comprises generating a hemoglobin concentration image of the patient's face and evaluating the hemoglobin concentration image using a machine learning model to determine the one or more first patient vital sign measurements.

12. The method of claim 1, wherein the one or more first patient vital sign measurements comprise one or more of patient blood pressure, patient body temperature, patient heart rate, patient respiratory rate, and patient blood oxygen level.

13. The method of claim 1, wherein transmitting the one or more first patient vital sign measurements and the second portion of the video feed to the medical professional communication device comprises causing the medical professional communication device to display the second portion of the video feed on a first portion of a screen of the medical professional communication device, and wherein transmitting the one or more first patient vital sign measurements comprises causing the medical professional communication device to display the one or more first patient vital sign measurements on a second portion of the screen of the medical professional communication device.

14. The method of claim 1, further comprising establishing, by the patient communication device, a connection with a family member communication device causing the family member communication device to join the video call.

15. A system, the system comprising: one or more hardware processors configured by machine-readable instructions to:
establish a video call with a medical professional communication device;
receive a first portion of a video feed of a patient's face from a camera of a patient communication device;
subsequent to receiving the first portion, receive a second portion of the video feed of the patient's face from the camera of the patient communication device;
process the first portion of the video feed from the camera of a patient communication device to determine one or more first patient vital sign measurements using one or more image processing techniques while transmitting the first portion of the video feed to the medical professional communication device; and
transmit the one or more first patient vital sign measurements and the second portion of the video feed to the medical professional communication device, receipt of the one or more first patient vital sign measurements causing the medical professional communication device to display the one or more first patient vital sign measurements concurrently with the second portion of the video feed,
wherein transmitting the one or more first patient vital sign measurements to the medical professional communication device comprises causing the medical professional communication device to display the one or more first patient vital sign measurements overlaid on the second portion of the video feed,
wherein the one or more first patient vital sign measurements are transmitted as raw numerical values or as a graphical representation

16. (canceled)

17. The system of claim 15, wherein transmitting the one or more first patient vital sign measurements comprises causing the medical professional communication device to display the one or more first patient vital sign measurements in real-time.

18. A non-transitory computer-readable storage medium having instructions embodied thereon, the instructions being executable by one or more processors to perform a method, the method comprising:
establishing a video call with a medical professional communication device;
receiving a first portion of a video feed of a patient's face from a camera of a patient communication device;
subsequent to receiving the first portion, receiving a second portion of the video feed of the patient's face from the camera of the patient communication device;
processing the first portion of the video feed from the camera of a patient communication device to determine one or more first patient vital sign measurements using one or more image processing techniques while transmitting the first portion of the video feed to the medical professional communication device; and
transmitting the one or more first patient vital sign measurements and the second portion of the video feed to the medical professional communication device, receipt of the one or more first patient vital sign measurements causing the medical professional communication device to display the one or more first patient vital sign measurements concurrently with the second portion of the video feed,
wherein transmitting the one or more first patient vital sign measurements to the medical professional communication device comprises causing the medical professional communication device to display the one or more first patient vital sign measurements overlaid on the second portion of the video feed,
wherein the one or more first patient vital sign measurements are transmitted as raw numerical values or as a graphical representation.

19. (canceled)

20. The non-transitory computer-readable storage medium of claim 18, wherein transmitting the one or more first patient vital sign measurements comprises causing the medical professional communication device to display the one or more first patient vital sign measurements in real-time.
